(19) United States
(12) Patent Application Publication
(10) Pub. No.: US 2022/0319647 A1
(43) Pub. Date: Oct. 6, 2022

(54) SYSTEMS AND METHODS FOR AN IMPROVED HEALTHCARE DATA FABRIC

(71) Applicant: P3 Health Partners, Henderson, NV (US)

(72) Inventors: James A. HENDERSON, Jr., Henderson, NV (US); Ajay CHAUDHARY, Cerritos, CA (US); Unmesh SRIVASTAVA, Monrovia, CA (US)

(73) Assignee: P3 Health Partners, Henderson, NV (US)

(21) Appl. No.: 17/657,163

(22) Filed: Mar. 30, 2022

Related U.S. Application Data
(60) Provisional application No. 63/169,377, filed on Apr. 1, 2021.
(60) Provisional application No. 63/169,393, filed on Apr. 1, 2021.

(51) Int. Cl. G16H 10/60; G06N 20/00

(52) U.S. Cl. CPC G16H 10/60; G06N 20/00

(57) ABSTRACT

A computer-implemented method for implementing a data fabric structure is disclosed. The method may comprise: receiving user data associated with a user from an external server via a secure network connection; storing the user data on a cloud-based data lake; transmitting the user data to a staging table; adding, to the staging table, user identification data and metadata; modifying the user data based on a determined correlation between the user data, the user identification data, and metadata to generate modified user data; comparing the modified user data and prior user data to determine a difference; upon determining the difference does not exceed a threshold, extracting, using a trained machine learning model, relevant data from the modified user data; formatting the relevant data into atomic data; generating a plurality of domains based on the atomic data, and presenting via a graphical user interface, graphical depictions of the plurality of domains.

### CROSS-REFERENCE TO RELATED APPLICATIONS

This application claims priority to U.S. Provisional Application No. 63/169,377 filed Apr. 1, 2021, and U.S. Provisional No. 63/169,393, filed Apr. 1, 2021, the entire disclosures of which are hereby incorporated herein by reference in their entireties.

### TECHNICAL FIELD

Various embodiments of this disclosure relate generally to data fabric structures for data analytics, and, more particularly, to systems and methods for implementing an improved healthcare data fabric structure for providing patient data analytics to healthcare providers and patients.

### BACKGROUND

Population health management is commonly understood as the process of improving clinical health outcomes for a defined group of individuals. For example, a population may be a specific age group (e.g., 55 years or older) residing in a specific region (e.g., Pima County, Arizona). Because different populations may have different health outcomes or different healthcare challenges, there exists a need to identify populations and determine common effective treatments and strategies for improving the population health. In particular, there is a need to provide tools and resources to patients in order to prevent, manage, and navigate illness. There further exists a need to assist providers by removing barriers or time-costs on providers, for example, by reducing or limiting administrative processes and “paper-pushing,” so that providers are able to spend more time focusing on patients.

With improvements in cloud computing, data storage technology, and data collecting applications, more data is available for processing now than ever before. In the context of healthcare, analysis of large volumes of patient data in particular is critical to assisting healthcare providers with making well-informed decisions and ultimately improving the quality of healthcare provided to patients. It is well-known that data analytics in healthcare is especially challenging in the United States, due to not only the large increase in the volumes of data being collected and stored, but due to lack of standardization of data formats, reporting, and applications that typically may vary between healthcare practices, hospitals, cities and states. This type of data analytics is especially important for identifying regions or communities that may not be receiving adequate or acceptable standards of care. For example, health outcomes, numbers of wellness visits, emergency room visits, and other metrics may be monitored for particular populations and compared to other populations. In this way, different populations may be compared, and populations who have lower health outcomes and quality of health service may be identified as needing additional assistance.

There are various disparate technologies attempting to address data management, especially in the healthcare context, including, for example, U.S. Pat. Pub. 2008/0208631A1 to Morita et al., U.S. Pat. Pub. No. 2015/0161331A1 to Mark Oleynik, and U.S. Pat. Pub. No. 2017/0364637A1 to Kshepakaran et al. However, conventional techniques, including the foregoing, fail to provide an improved and effective data fabric structure for providing data analytics to providers and patients.

The background description provided herein is for the purpose of generally presenting the context of the disclosure. Unless otherwise indicated herein, the materials described in this section are not prior art to the claims in this application and are not admitted to be prior art, or suggestions of the prior art, by inclusion in this section.

### SUMMARY OF THE DISCLOSURE

According to certain aspects of the disclosure, methods and systems are disclosed for implementing a data fabric structure.

In one aspect, an exemplary embodiment of a computer-implemented method for implementing a data fabric structure may include: receiving user data associated with a user from an external server via a secure network connection; storing the user data on a cloud-based data lake; transmitting the user data to a staging table; adding to the staging table, user identification data and metadata; modifying the user data based on a determined correlation between the user data, the user identification data, and metadata to generate modified user data; extracting relevant data from the modified user data; formatting the relevant data into atomic data; generating a plurality of domains based on the atomic data; and presenting, via a graphical user interface, one or more graphical depictions of data associated with the plurality of domains.

In another aspect, an exemplary embodiment of a computer-implemented method for using a trained machine-learning model for implementing a data fabric structure may include: receiving user data associated with a user from an external server via a secure network connection; storing the user data on a cloud-based data lake; transmitting the user data to a staging table; adding to the staging table, user identification data and metadata; modifying the user data based on a determined correlation between the user data, the user identification data, and metadata to generate modified user data; comparing the modified user data and prior user data to determine a difference between the modified user data and the prior user data; upon determining that the difference does not exceed a predetermined threshold: extracting, using a trained machine learning model, relevant data from the modified user data, wherein the trained machine learning model is trained to extract relevant data from the modified user data based on (i) training relevancy data that includes information regarding prior relevant data extracted from prior modified user data associated with other users and (ii) training user data that includes prior relevant data extracted from prior modified user data, to learn relationships between the training relevancy data and the training user data, such that the trained machine learning model is configured to use the learned relationships to extract modified user data in response to input of the modified user data; upon determining that the difference does exceed a predetermined threshold, automatically, by the one or more processors: removing the modified user data from the staging table; transmitting an error notification to an entity associated with the external server; and extracting relevant data from the prior user data; formatting the relevant data into atomic data; generating a plurality of domains and sub-domains based on the atomic data; and displaying, via a graphical user interface, one or more graphical depictions of data associated with the plurality of domains and sub-domains.

In a further aspect, an exemplary embodiment of a system for implementing a data fabric structure may include: a memory storing instructions; and a processor operatively connected to the memory and configured to execute the instruction to perform operations and/or processes. The process may include: receiving user data associated with a user from an external server via a secure network connection; storing the user data on a cloud-based data lake; transmitting the user data to a staging table; adding, to the staging table, user identification data and metadata; modifying the user data based on a determined correlation between the user data, the user identification data, and metadata to generate modified user data; extracting relevant data from the modified user data; formatting the relevant data into atomic data; generating a plurality of domains based on the atomic data; presenting, via a graphical user interface, one or more graphical depictions of data associated with the plurality of domains.

It is to be understood that both the foregoing general description and the following detailed description are exemplary and explanatory only and are not restrictive of the disclosed embodiments, as claimed.

### BRIEF DESCRIPTION OF THE DRAWINGS

The accompanying drawings, which are incorporated in and constitute a part of this specification, illustrate various exemplary embodiments and together with the description, serve to explain the principles of the disclosed embodiments.

FIG. 1 depicts an exemplary environment for implementing a data fabric structure, according to one or more embodiments.

FIG. 2 depicts a representation of an exemplary implementation of a data fabric structure, according to one or more embodiments.

FIG. 3 depicts a flowchart of an exemplary method of implementing a data fabric structure, according to one or more embodiments.

FIG. 4 depicts an exemplary graphical user interface implemented as a physician portal, according to one or more embodiments.

FIG. 5 depicts a flowchart of an exemplary method of using a trained machine-learning model to implement a data fabric structure, according to one or more embodiments.

FIG. 6 depicts an example of a computing device, according to one or more embodiments.

### DETAILED DESCRIPTION OF EMBODIMENTS

According to certain aspects of the disclosure, methods and systems are disclosed for implementing a data fabric structure, e.g., a data fabric structure for healthcare data. There is a need to provide tools and resources for patients and healthcare providers to improve population health. However, conventional techniques may not be suitable. For example, conventional techniques may not provide an improved data fabric structure that efficiently provides data to. Accordingly, improvements in technology relating to data fabric structures are needed.

As will be discussed in more detail below, in various embodiments, systems and methods are described for using machine learning to extract relevant data from patient data stored on a data lake. By training a machine-learning model, e.g., via supervised or semi-supervised learning, to learn associations between training relevancy data that includes information regarding prior relevant data extracted from prior modified user data associated with other users and training user data that includes prior relevant data extracted from prior modified user data, the trained machine-learning model may be usable to extract relevant data in response to input of the modified user data.

Reference to any particular activity is provided in this disclosure only for convenience and not intended to limit the disclosure. A person of ordinary skill in the art would recognize that the concepts underlying the disclosed devices and methods may be utilized in any suitable activity. The disclosure may be understood with reference to the following description and the appended drawings, wherein like elements are referred to with the same reference numerals.

The terminology used below may be interpreted in its broadest reasonable manner, even though it is being used in conjunction with a detailed description of certain specific examples of the present disclosure. Indeed, certain terms may even be emphasized below; however, any terminology intended to be interpreted in any restricted manner will be overtly and specifically defined as such in this Detailed Description section. Both the foregoing general description and the following detailed description are exemplary and explanatory only and are not restrictive of the features, as claimed.

In this disclosure, the term “based on” means “based at least in part on.” The singular forms “a,” “an,” and “the” include plural referents unless the context dictates otherwise. The term “exemplary” is used in the sense of “example” rather than “ideal.” The terms “comprises,” “comprising,” “includes,” “including,” or other variations thereof, are intended to cover a non-exclusive inclusion such that a process, method, or product that comprises a list of elements does not necessarily include only those elements, but may include other elements not expressly listed or inherent to such a process, method, article, or apparatus. The term “or” is used disjunctively, such that “at least one of A or B” includes, (A), (B), (A and A), (A and B), etc. Relative terms, such as, “substantially,” “approximately,” and “generally,” are used to indicate a possible variation of ±10% of a stated or understood value.

It will also be understood that, although the terms first, second, third, etc. are, in some instances, used herein to describe various elements, these elements should not be limited by these terms. These terms are only used to distinguish one element from another. For example, a first contact could be termed a second contact, and, similarly, a second contact could be termed a first contact, without departing from the scope of the various described embodiments. The first contact and the second contact are both contacts, but they are not the same contact.

As used herein, the term “if” is, optionally, construed to mean “when” or “upon” or “in response to determining” or “in response to detecting,” depending on the context. Similarly, the phrase “if it is determined” or “if [a stated condition or event] is detected” is, optionally, construed to mean “upon determining” or “in response to determining” or “upon detecting [the stated condition or event]” or “in response to detecting [the stated condition or event],” depending on the context.

Terms like “provider,” “merchant,” “vendor,” or the like generally encompass an entity or person involved in providing, selling, and/or renting items to persons such as a seller, dealer, renter, merchant, vendor, or the like, as well as an agent or intermediary of such an entity or person. An “item” generally encompasses a good, service, or the like having ownership or other rights that may be transferred. As used herein, terms like “user” or “customer” generally encompasses any person or entity that may desire information, resolution of an issue, purchase of a product, or engage in any other type of interaction with a provider. The term “browser extension” may be used interchangeably with other terms like “program,” “electronic application,” or the like, and generally encompasses software that is configured to interact with, modify, override, supplement, or operate in conjunction with other software. As used herein, terms such as “user data” or the like generally encompass patient data, or data pertaining to one or more medical patients. A “staging table” generally refers to a permanent database, data structure, data tables, or the like used to store temporary data for future processing. “Atomic data” generally refers to data in a data store, database or data warehouse that is at its lowest level of detail, e.g., data that cannot be broken down into smaller parts (e.g., a zip code may be considered “Atomic Data” because it cannot be broken down any further into another data element).

As used herein, a “machine-learning model” generally encompasses instructions, data, and/or a model configured to receive input, and apply one or more of a weight, bias, classification, or analysis on the input to generate an output. The output may include, for example, a classification of the input, an analysis based on the input, a design, process, prediction, or recommendation associated with the input, or any other suitable type of output. A machine-learning model is generally trained using training data, e.g., experiential data and/or samples of input data, which are fed into the model in order to establish, tune, or modify one or more aspects of the model, e.g., the weights, biases, criteria for forming classifications or clusters, or the like. Aspects of a machine-learning model may operate on an input linearly, in parallel, via a network (e.g., a neural network), or via any suitable configuration.

The execution of the machine-learning model may include deployment of one or more machine learning techniques, such as linear regression, logistical regression, random forest, gradient boosted machine (GBM), deep learning, and/or a deep neural network. Supervised and/or unsupervised training may be employed. For example, supervised learning may include providing training data and labels corresponding to the training data, e.g., as ground truth. Unsupervised approaches may include clustering, classification or the like. K-means clustering or K-Nearest Neighbors may also be used, which may be supervised or unsupervised. Combinations of K-Nearest Neighbors and an unsupervised cluster technique may also be used. Any suitable type of training may be used, e.g., stochastic, gradient boosted, random seeded, recursive, epoch or batch-based, etc.

In an exemplary use case, external patient data may be obtained from an external database associated with one or more health institutions, insurance companies, and/or healthcare providers (e.g., Cigna, Aetna, Anthem, Blue Cross Blue Shield, and so forth) and stored on a cloud-based data lake associated with a data fabric system, for example, Microsoft Azure Data Lake. An internal database for the data fabric system may also hold other patient-related information, for example, data obtained from a third party claims-processing company such as Citra Health Solutions, information related to a patient's claims or payments. Patient data from the cloud-based data lake may and the internal database may then be imported to a staging table where it is prepared for integration. At this step, patient data may be modified, for example, by modifying the data into a better format for aggregation. As another example, data for a particular patient may be associated with multiple different member IDs; in that situation, the patient data may be parsed and mapped to a preferred member ID for easier reference downstream. After the patient data is modified, relevant information may then be extracted from the modified data. For example, patient contact information might be considered relevant, and this data may be extracted. Similarly, information related to a recent hospital visit (visit date, discharge date and time, diagnoses, prescriptions, testing scheduled, and so forth) may each be found relevant and also extracted from the modified data. In some cases, a trained machine learning model may be trained to extract the data. In some embodiments, exception handling may be performed; for example, if it is clear that there is an error in the data (for example, there are 5000 new patients being added to the system during a routine update), the system may determine that a threshold is exceeded such that the data should be rejected. In that case, instead of loading new data, previously used or “old” data may temporarily be used until new data is received. The relevant data may then be extracted and parsed to generate atomic flattened data. This atomic data is then classified into one or more domains or subdomains. For example, the atomic data may be placed into the lab results domain, or the medical claim domain. Because the data has been audited and sorted into a plurality of domains and subdomains, it is now easier for one or more product schemas or business reporting analytics to access the integrated data for display on a graphical user interface. This data may be utilized in a variety of ways; for example, by consolidating all this data quickly and efficiently, a provider can more quickly view and access various healthcare provider tools. Further, data pertaining to one or more populations may now be quickly and accurately accessed, for example, via a web and/or mobile application based portal. Using this data, at-risk communities can be identified for specialized healthcare treatment and programs.

While the example above involves patient or healthcare data, it should be understood that techniques according to this disclosure may be adapted to any suitable type of data. It should also be understood that the examples above are illustrative only. The techniques and technologies of this disclosure may be adapted to any suitable activity.

Presented below are various aspects of machine learning techniques that may be adapted to extract relevant patient data in response to input of the modified user patient data. As will be discussed in more detail below, machine learning techniques adapted to extract relevant data in response to input of the modified user data, may include one or more aspects according to this disclosure, e.g., a particular selection of training data, a particular training process for the machine-learning model, operation of a particular device suitable for use with the trained machine-learning model, operation of the machine-learning model in conjunction with particular data, modification of such particular data by the machine-learning model, etc., and/or other aspects that may be apparent to one of ordinary skill in the art based on this disclosure.

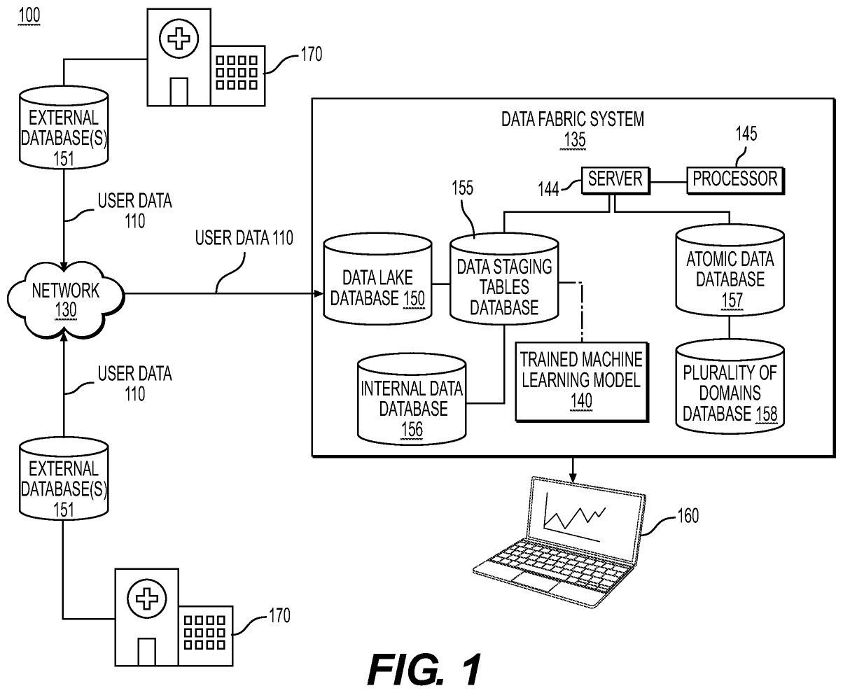

FIG. 1 depicts an exemplary environment, such as environment 100, which may be utilized with techniques presented herein. One or more healthcare institutions 170, external database(s) 151, and graphical user interface 160 (“GUI”) may communicate across an electronic network 130. As will be discussed in further detail below, one or more data fabric systems, for example, data fabric system 135, may communicate with one or more of the other components of the environment 100 across electronic network 130. The data fabric system 135 may according to some aspects of this disclosure comprise a processor 145, a server 144, a data staging tables database 155, a data lake database 150, an internal data database 156, a trained machine learning model 140, an atomic data database 157, and a plurality of domains database 158. The data lake database 150 may be a cloud-based database, and according to some aspects, may be located separately from the data fabric system 135. The graphical user interface 160 may be associated with a health care provider or a user, e.g., a user associated with one or more of generating, training, or tuning a machine-learning model for implementing a data fabric system, generating, obtaining and/or analyzing user data (e.g., patient healthcare data).

In some embodiments, the components of the environment 100 are associated with a common entity, e.g., a healthcare institution, a healthcare insurer, a population health management company, or the like. In some embodiments, one or more of the components of the environment is associated with a different entity than another. The systems and devices of the environment 100 may communicate in any arrangement. As will be discussed herein, systems and/or devices of the environment 100 may communicate in order to one or more of generate, train, or use a machine-learning model to implement a data fabric system, among other activities.

The graphical user interface 160 may be configured to enable a health care provider or user to access and/or interact with other systems in the environment 100. For example, the graphical user interface 160 may be implemented as a web and/or mobile application based portal on a computer system such as, for example, a desktop computer, a mobile device, a tablet, etc. In some embodiments, the graphical user interface 160 may be associated or implemented by a user device that may include one or more electronic application(s), e.g., a program, plugin, browser extension, etc., installed on a memory of the user device. In some embodiments, the electronic application(s) may be associated with one or more of the other components in the environment 100. For example, the electronic application(s) may include one or more of system control software, system monitoring software, software development tools, etc.

The user device associated with the graphical user interface 160 may include a server system, an electronic medical data system, computer-readable memory such as a hard drive, flash drive, disk, etc. In some embodiments, the user device associated with graphical user interface 160 includes and/or interacts with an application programming interface for exchanging data to other systems, e.g., one or more of the other components of the environment. The user device may include and/or act as a repository or source for user data/patient related data. For example, data from Citra software provided by Citra Health Solutions, Inc., as discussed in more detail below.

In various embodiments, the electronic network 130 may be a wide area network (“WAN”), a local area network (“LAN”), personal area network (“PAN”), or the like. In some embodiments, electronic network 130 includes the Internet, and information and data provided between various systems occurs online. “Online” may mean connecting to or accessing source data or information from a location remote from other devices or networks coupled to the Internet. Alternatively, “online” may refer to connecting or accessing an electronic network (wired or wireless) via a mobile communications network or device. The Internet is a worldwide system of computer networks-a network of networks in which a party at one computer or other device connected to the network can obtain information from any other computer and communicate with parties of other computers or devices. The most widely used part of the Internet is the World Wide Web (often-abbreviated “VWWV” or called “the Web”). A “website page” generally encompasses a location, data store, or the like that is, for example, hosted and/or operated by a computer system so as to be accessible online, and that may include data configured to cause a program such as a web browser to perform operations such as send, receive, or process data, generate a visual display and/or an interactive interface, or the like.

As discussed in further detail below, the data fabric system 135 may one or more of (i) generate, store, train, or use a machine-learning model configured to extract relevant information from modified user data. The data fabric system 135 may include a machine-learning model and/or instructions associated with the machine-learning model, e.g., instructions for generating a machine-learning model, training the machine-learning model, using the machine-learning model etc. The data fabric system 135 may include instructions for retrieving user data 110, adjusting user data 110, e.g., based on the output of the machine-learning model, and/or operating graphical user interface 160 to output relevant data or atomic data, e.g., as adjusted based on the machine-learning model. The data fabric system 135 may include training data, e.g., training relevancy data that includes information regarding prior relevant data extracted from prior modified user data associated with other users, and may include ground truth, e.g., training user data that includes prior relevant data extracted from prior modified user data.

In some embodiments, a system or device other than the data fabric system 135 is used to generate and/or train the machine-learning model. For example, such a system may include instructions for generating the machine-learning model, the training data and ground truth, and/or instructions for training the machine-learning model. A resulting trained-machine-learning model may then be provided to the data fabric system 135.

Generally, a machine-learning model includes a set of variables, e.g., nodes, neurons, filters, etc., that are tuned, e.g., weighted or biased, to different values via the application of training data. In supervised learning, e.g., where a ground truth is known for the training data provided, training may proceed by feeding a sample of training data into a model with variables set at initialized values, e.g., at random, based on Gaussian noise, a pre-trained model, or the like. The output may be compared with the ground truth to determine an error, which may then be back-propagated through the model to adjust the values of the variable.

Training may be conducted in any suitable manner, e.g., in batches, and may include any suitable training methodology, e.g., stochastic or non-stochastic gradient descent, gradient boosting, random forest, etc. In some embodiments, a portion of the training data may be withheld during training and/or used to validate the trained machine-learning model, e.g., compare the output of the trained model with the ground truth for that portion of the training data to evaluate an accuracy of the trained model. The training of the machine-learning model may be configured to cause the machine-learning model to learn associations between training relevancy data that includes information regarding prior relevant data extracted from prior modified user data associated with other users and training user data that includes prior relevant data extracted from prior modified user data, such that the trained machine-learning model is configured to determine an output relevant data in response to the input modified user data based on the learned associations.

In various embodiments, the variables of a machine-learning model may be interrelated in any suitable arrangement in order to generate the output. For example, in some embodiments, the machine-learning model may include image-processing architecture that is configured to identify, isolate, and/or extract features, geometry, and or structure in one or more of the medical imaging data and/or the non-optical in vivo image data. In some instances, different samples of training data and/or input data may not be independent. Thus, in some embodiments, the machine-learning model may be configured to account for and/or determine relationships between multiple samples.

Although depicted as separate components in FIG. 1, it should be understood that a component or portion of a component in the environment 100 may, in some embodiments, be integrated with or incorporated into one or more other components. For example, a portion of the graphical user interface 160 may be integrated into one or multiple user devices or the like. In another example, the data fabric system 135 may be integrated a data storage system. In some embodiments, operations or aspects of one or more of the components discussed above may be distributed amongst one or more other components. Any suitable arrangement and/or integration of the various systems and devices of the environment 100 may be used.

Further aspects of the machine-learning model and/or how it may be utilized to implement a data fabric structure are discussed in further detail in the methods below. In the following methods, various acts may be described as performed or executed by a component from FIG. 1, such as the data fabric system 135 or components thereof. However, it should be understood that in various embodiments, various components of the environment 100 discussed above may execute instructions or perform acts including the acts discussed below. An act performed by a device may be considered to be performed by a processor, actuator, or the like associated with that device. Further, it should be understood that in various embodiments, various steps may be added, omitted, and/or rearranged in any suitable manner.

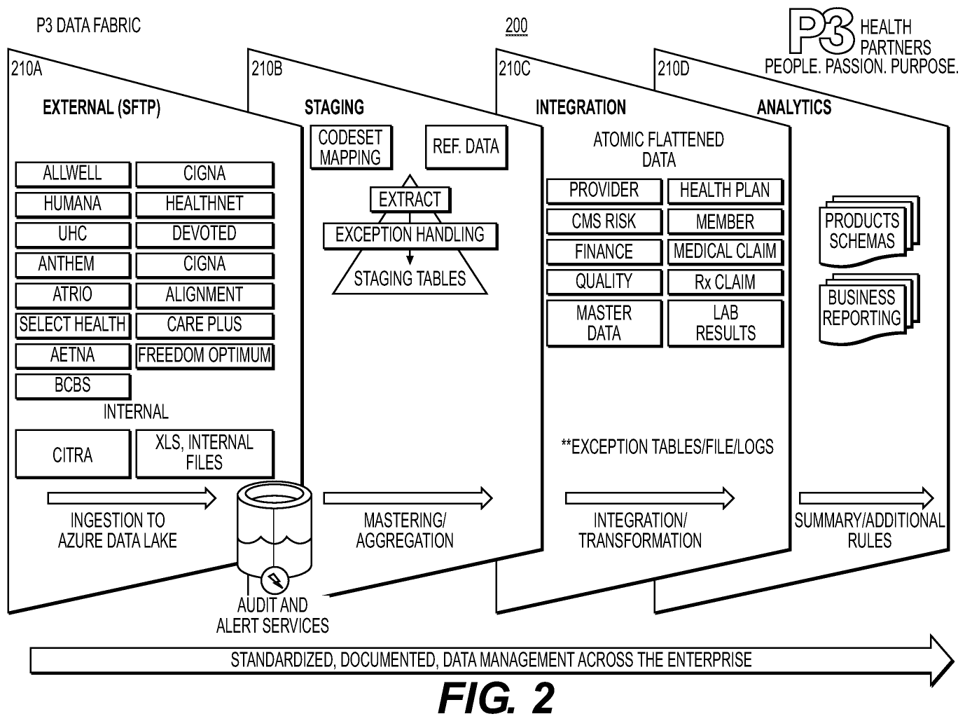

FIG. 2 illustrates a representation of an exemplary implementation of a data fabric structure 200, according to one or more aspects of the disclosure. As shown in FIG. 2, at 210A, user data may be received via a secure file transfer process from external databases, such as a secure hypertext transfer protocol (S-HTTP), and stored on a data lake, for example a cloud-based data lake such a Microsoft Azure Data Lake. The external databases may be associated with one or more health institutions or insurers as depicted (e.g., Allwell, Humana, Cigna, Healthnet, and so forth). The user data may comprise information related to a patient, for example, hospital visits, emergency room visits, drug prescriptions, insurance information, claims information, diagnoses, primary care doctors, medical expenses, and so forth. Additional data from an internal server may also be stored on the data lake, such as Citra data or other relevant internal data relevant to a patient, for example, patient payment information or credit score information. Data stored on the data lake may be stored in its original format, e.g., .xls, .csv, text, pipe delimited, and other typical format. Once user data (e.g. patient data) is collected on the data lake, it may then be sent to a staging process, where the data is temporarily placed into one or more staging tables at 210B. Once user data is received at the staging tables, it is processed for integration. While the data at the staging table in this embodiment is kept in its original format, in some embodiments, the data may be formatted into another file format. Once the data is placed on the table, it may be processed or modified. For example, as explained above, data on the table may be mapped to other data received from an internal database or other source. For example, the source data may have multiple different member IDs for the same patient, but the data fabric relies on only one type of member ID. The user data may thus be modified and mapped to the optimal member ID for further processing. Relevant data may then be extracted from the modified user data. Further, during this process, the data may be audited. For example, if the data indicates that 5000 new patients are being added for a particular health care provider, the significant change in size may indicate an error in the received data. When an error is detected, the new data may be rejected. In some cases, upon auditing detecting an error, previously used relevant data or a version thereof may be used. In this manner, a previously saved version of the relevant data will remain accessible to a healthcare provider and data analytics downstream even if the newly received data from the data lake contains errors. Once relevant user data has been extracted, at 210 C, the relevant data may be further parsed at integration to generate atomic flattened data, e.g., data that has been broken down into its smallest logical unit. For example, data such as a price, a zip code, or a street address number may be atomic data. On the other hand, data such as an entire street address (which has multiple fields) would not be considered atomic data. Once the atomic data has been obtained, it may then be sorted into one or more domains. Domains may include, for example, a provider domain, lab results domain, medical claim domain, member domain, and so forth. By sorting the atomic data into a plurality of domains, the data can now be easily and quickly retrieved by one or more analytics processes, for example, a products scheme or business reporting at 210D. In this manner, and improved data fabric structure is provided that results in a demonstrably improved software for healthcare providers and patients.

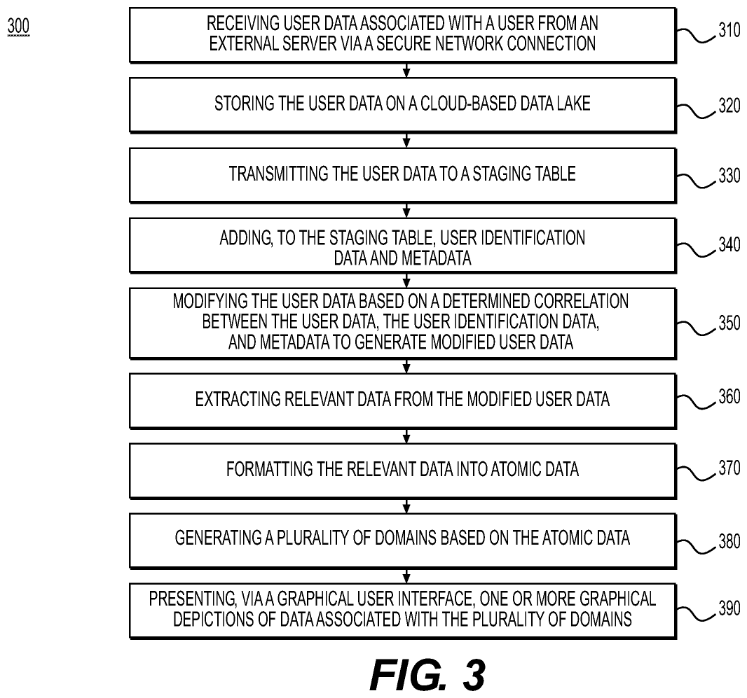

FIG. 3 illustrates a flowchart of an exemplary method of implementing a data fabric structure, such as in the various examples discussed above. At step 310 of the flowchart 300, the data fabric system 135 may receive user data 110 associated with a user from an external database, for example external database(s) 151 associated with a healthcare institution 170, via a secure network connection such as SSH (secure shell protocol)/secure file transfer protocol (SFTP), hypertext transfer protocol secure (HTTPS), secure hypertext transfer protocol (S-HTTP), and so forth. According to some aspects, the user data 110 comprises one or more of: user institution records, user identification information, or user financial data. User data 110 may be, for example, patient data, including data relevant to a patient or user's health medical records or history. For example, user data 110 may include medical records such as diagnoses, lab test results, x-ray results, emergency room visit and discharge records, hospital or clinic admission information, and other information relevant to the health, medical treatment, and well-being of a user or patient. According to some aspects, the user data 110 is in the format of one or more of: an xIs file; a csv file; a pipe delimited file, or a text file. At step 320, the user data 110 may be stored on a data lake, such as data lake database 150 and/or a cloud-based data lake such as Microsoft Azure Data Lake. A data lake according to some aspects is a repository of data that can store a large amount of raw (e.g., unprocessed data, structured data, unstructured data, etc.) data in its native format. For example, an xIs file received from Cigna would be stored on the data lake in an xIs format, without any modification of formatting of the data.

At step 330, the user data 110 or portions thereof may be transmitted to a staging table, for example, data staging tables database 155. According to some aspects, the user data 110 is transmitted to the staging table again in its raw (e.g. unprocessed) format. At step 340, the data fabric system 135 may process the user data 110 by adding user identification data and metadata, for example, data obtained from internal data database 156, to the data staging tables database 155. According to some aspects, the metadata may include a time stamp associated with the time the user data was stored on the cloud-based data lake. In some aspects, the data may be claims processing data, for example, data obtained from Citra software pertaining to a patient's medical patient's claim records, payment information, or to medical codes associated with a patient's medical care. In some embodiments, the data received or obtained from Citra software may be stored on the data lake database 150. According to some aspects, the metadata added to the staging table (e.g. data staging tables database 155) may include a date the data was loaded onto the data lake database 150 or the data staging tables database 155.

At step 350, the data fabric system 135 may modify the user data based on a determined correlation between the user data, the user identification data, and metadata to generate modified user data. According to some aspects, the internal data may include a mapping file for member identification. For example, a user patient identification (ID) contained in the user data 110 loaded to the staging table may be different from a member ID on file or stored on an internal database or with the data fabric system 135. According to some aspects, a mapping file mapping the user data 110 to the member ID stored on the internal data database 156. In this manner, user data 110 may be modified or formatted to generate modified user data that is mapped to the internal member ID and includes metadata indicating a time of upload. According to some aspects, this modified data may be generated using an extract, transform and load (ETL) process on the user data 110. In this manner, the user data 110 is modified to make extraction of relevant information easier as explained further below at step 360.

At step 360, the data fabric system 135 may extract relevant data from the modified user data. Relevant data may be, for example, data that has previously been used or requested by downstream analytics processes. For example, relevant data may include a user's name, address, contact information, number of hospital or emergency room visits over a time frame, associated medical or claim codes, and other discrete portions of information that may be helpful for data analytics processes. According to some aspects of the disclosure, the relevant data may be extracted from the modified user data using a trained machine learning model. According to this aspect of the disclosure, the trained machine learning model may be trained to extract relevant data from the modified user data based on (i) training relevancy data that includes information regarding prior relevant data extracted from prior modified user data associated with other users and (ii) training user data that includes prior relevant data extracted from prior modified user data, to learn relationships between the training relevancy data and the training user data, such that the trained machine learning model is configured to use the learned relationships to extract modified user data in response to input of the modified user data.

At step 370, the data fabric system 135 may format the relevant data into atomic data, and subsequently store that data on a database, for example, atomic data database 157. Atomic data refers to data that typically cannot be broken down into smaller parts. For example, a postal code or zip code, a street address number, a medical claim code, gross revenue, a base salary, unit sales for a product, a username, a password, and so forth. Thus, according to some aspects, all relevant information extracted from the modified user data is separated into atomic data and stored.

At step 380, the data fabric system 135 may generate a plurality of domains based on the atomic data and subsequently store the plurality of domains on plurality of domains database 158. A domain as used herein refers to a collection of values that a data element may contain. For example, a domain may be “lab results,” which may comprise elements including “lab name,” “test name,” “test code,” “test cost,” and “test results.” According to some aspects of the disclosure, one of the plurality of domains may further include one or more sub-domains. For example, “test results” may be a sub-domain, which may further have additional information such as “positive” “negative” or “inclusive.” According to aspects of the disclosure, the plurality of domains may include one or more of: a provider domain; a cms domain; a risk domain; a finance domain; a quality domain; a master data domain; a health plan domain; a member domain; a medical claim domain; a T 1 claim domain; or a lab result domain. According to some aspects of the disclosure, each domain of the plurality of domains may be stored in an SQL file format. By using atomic data to populate the plurality of domains, only information necessary to a particular domain is extracted from the data. Further, a single file format, for example, SQL file format, may be used across the domains. By formatting the user data 110 into atomic data and sorting that data into domains, downstream product schemas and other software can more easily and quickly pull relevant data for data analytics processes.

At step 390, the data fabric system 135 may cause to present, via a graphical user interface such as graphical user interface 160, one or more graphical depictions of data associated with the plurality of domains. For example, the graphical user interface 160 may include a healthcare provider or physician dashboard, and include data pertaining to a total number of members or patients, a percent of members who have completed wellness visits, a number of patients currently in ER, and so forth. The number of patients may also be depicted with breakdowns of the type of members, for example, members who are considered senior or over the age of 50+ or new members. Providing this data to a physician in this manner can provide the physicians with tools that allow a doctor to quickly review patient data and see trends or determine who needs to schedule an appointment without the need to conduct file by file reviews or hire staff to conduct such reviews. Graphical user interface 160 may be configured such that icons and/or components are dynamically determined for display, via graphical user interface 160, based on available domains for a given patient or patient population. The icons and/or components may be dynamically determined based on data identified to be displayed via graphical user interface 160. The data may be identified based on available domain data, based on user request for given data, and/or based on a machine learning output. The data may also or alternatively be audit checked prior to updating GUI 160 to prevent any issues with the upstream data (e.g., an insurance company or healthcare provider system being offline) from reducing the usefulness of GUI 160. If the audit check clearly indicates an error in the data provided for updating GUI 160, the system may determine that an error threshold is exceeded such that the GUI should not be updated with the incomplete and/or erroneous data.

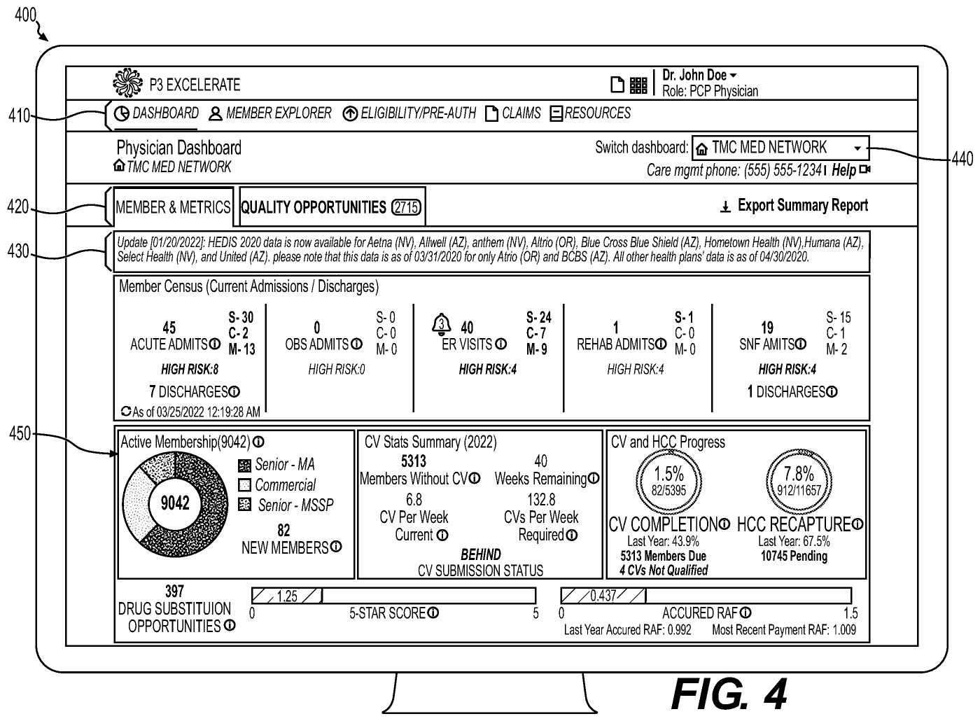

FIG. 4 illustrates an exemplary graphical user interface 400 implemented as, for example, a physician portal. For example, GUI 400 may represent an exemplary user interface that a care provider may see when logging into a physician dashboard. GUI 400 may be accessed via a browser displayed webpage or an application interface, for example, on a mobile, tablet, or desktop device.

Portal header 410 can identify a number of portal views such as a dashboard view, a member specific view, an eligibility/pre-authorization view, a claims view, and a view to provide other resources. The option selected from portal header 410 can determine the information displayed on GUI 400 and/or the manner in which the information is organized and/or visualized. Below portal header 410, GUI 400 may include a series of additional elements, such as tabs 420, notification(s) 430, drop down menu(s) 440, data visualization(s) 450, and/or other static or interactive elements to help a user view, input, or edit desired information. For example, GUI 400 can allow a user to view and sort patients by status and/or care level required/provided (e.g., acute, under observation, emergency department, skilled nursing facility, rehab). Users may also be able to generate simplified visualizations of select portions of the data fabric, such as active caseload, patients due for care, potential drug substitution opportunities, suspected conditions (generated by reviewers and/or trained machine learning model 140), and/or other user or department specific information. As depicted in FIG. 4, GUI 400 can include information, for example, regarding how many members are without data and how many weeks are left in a current year. Based on member submission rates in comparison to one or more required rates, an overall status may be provided to track whether a member is ahead, on track, or behind based on the weeks left in the current year.

In some embodiments, GUI 400 can also serve as a portal for document and data entry by providing options for documents or other information to be uploaded, via GUI 400, to be fed back into data lake database 150 and/or external databases 151. GUI 400, when implemented as a physician and/or patient portal, may enable communication between providers, patients, medical labs, insurers, and other parties involved in providing or supporting care. By enabling access to data in a comprehensive and intuitive manner, GUI 400, in combination with the disclosed data fabric structural concepts, can help close existing care gaps.

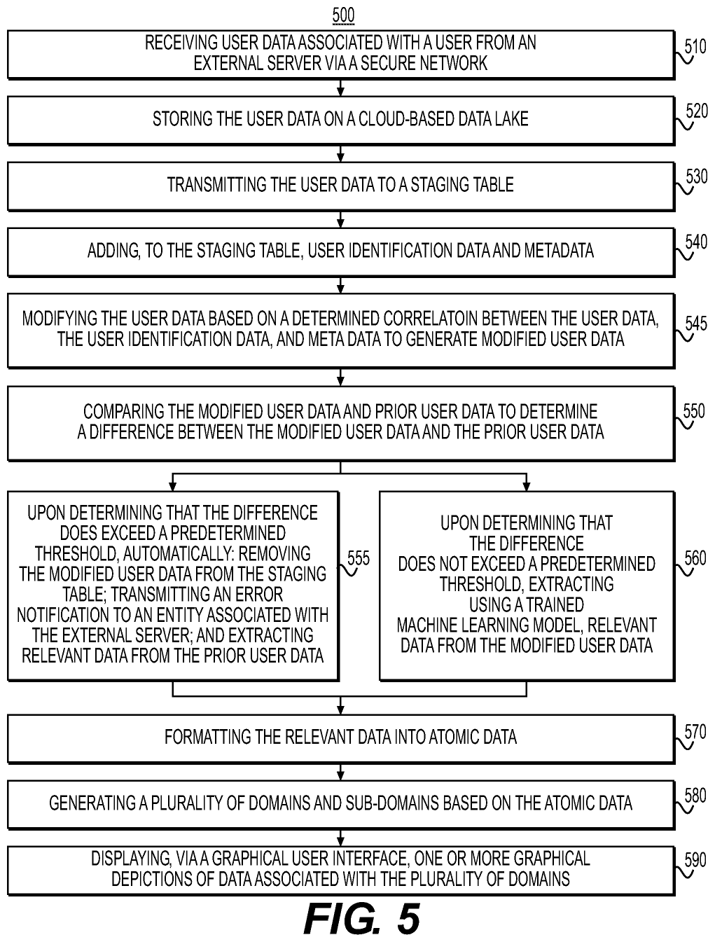

FIG. 5 illustrates a flowchart of an exemplary method of using a trained machine-learning model to implement a data fabric structure. At step 510 of the flowchart 500, the data fabric system 135 may receive (e.g., S-HTTP) user data 110 associated with a user from an external database as described above with respect to step 310 of FIG. 3. According to some aspects of the disclosure, the external server is associated with a health insurance company or a hospital. At step 520, the user data 110 may be stored on a data lake as described above with respect to step 320 of FIG. 3. At step 530, the user data 110 or portions thereof may be transmitted to a staging table as described above with respect to step 330 of FIG. 3. At step 540, the data fabric system 135 may add user identification data and metadata (e.g., a time stamp associated with the time the user data was stored on the cloud-based data lake) to a data staging table as described above with respect to step 340 of FIG. 3. At step 545, the data fabric system 135 may modify the user data based on a determined correlation between the user data, the user identification data, and metadata to generate modified user data as described above with respect to step 350 of FIG. 3.

At step 550, the data fabric system 135 may compare the modified user data and prior user data to determine a difference between the modified user data and the prior user data. For example, a physician or healthcare provider may be associated with a certain number of patients or users, for example, 700 patients. When user data 110 is uploaded for processing, a review of the data may indicate that the physicians might have 5000 new members. The data fabric system 135 may determine that a threshold is exceeded, such that there is an indication of an error with the data. As another example, user data 110 may indicate that $2,000,000 in claims were paid, but the prior data indicates that only $25,000 was previously paid. Depending on the threshold, which may be predetermined or assigned, the data fabric system 135 may determine whether the amount or member numbers in the modified user data exceeds a threshold based on the prior user data. For example, a predetermined threshold might be $10,000 in the context of claims payments, or it might be 50 patients or members in the context of member numbers. A relatively large difference may thus indicate a likelihood of error that may be detected by the data fabric system 135.

At step 555, upon determining that the difference does exceed a predetermined threshold, the data fabric system 135 may automatically remove the modified user data from the staging table, transmit an error notification to an entity associated with the external server, and extract relevant data from the prior user data. For example, upon determining an error or threshold is met, a health insurer may be notified that user data 110 sent to the data fabric system 135 is likely erroneous, and to request corrected user data. In some embodiments, as explained above, the modified user data may include a quantity indicating a number of members associated with a provider, and accordingly, the modified user data and the prior user data may be compared by comparing a number of members associated with the provider and a prior number of members associated with the provider. According to additional aspects, the modified user data may include an amount of paid claims associated with a provider, and comparing the modified user data and the prior user data includes comparing the amount of paid claims associated with the provider and a prior amount of paid claims associated with the provider. As another example, the user data 110 may be audited to determine if information is missing. For example, if no ID information is included with the user data 110, this may also indicate an error that the data fabric system 135 may detect, and then use to reject the user data 110.

According to some aspects, the data fabric system 135 may compare the modified user data and prior user data to determine a difference between the modified user data and the prior user data. For example, the modified user data may indicate 1,000 members, but the prior user data indicates that there are 10,000 members. If a predetermined threshold is set to 30%, because the difference in membership represents a 90% decrease in membership, the data fabric system 135 may determine that the difference between the modified user data and prior user data (90% decrease) exceeds the predetermined threshold (30%). While a percentage is used here, other measurements may be used, including for example a ratio or a numerical difference (for example, a predetermined threshold may be $10,000 or 150 members), and/or dynamic thresholds based on time of year (e.g., higher thresholds during open enrollment periods), member growth projections (e.g., based on acquisitions/mergers between insurers, providers, or health systems), or the like. Upon determining that a difference exceeds a predetermined threshold, the data fabric system 135 may automatically remove the modified user data from the staging table, refrain from extracting relevant data from the modified user data, transmit an error notification to an entity associated with the external server, and proceed to load and extract relevant data from the prior user data. For example, prior user data may be stored on an internal database, such as internal data database 156. In the event that user data 110 received is incorrect, data should still be supplied to downstream analytics software for the physician or provider. As such, a previous version of the data, such as prior data or previously used modified data, may be sent to the staging table. In this manner, the graphical user interface 160 will still receive accurate, albeit older, versions of data. In this manner, the most up to date and accurate patient information or user data is still provided to the health provider even if erroneous data is detected or audited and removed from the data fabric system 135. According to some aspects of the disclosure, steps 550 and 555 may be automated or automatically performed by the data fabric system 135.

At step 560, the data fabric system 135 may, upon determining that the difference does not exceed a predetermined threshold, extract, using a trained machine learning model, for example trained machine learning model 140, relevant data from the modified user data as described above with respect to step 360 of FIG. 3. At step 570, the data fabric system 135 may format the relevant data into atomic data as described above with respect to step 370 of FIG. 3. At step 580, the data fabric system 135 may generate a plurality of domains based on the atomic data as described above with respect to step 380 of FIG. 3. A plurality of subdomains may be generated for one of the plurality of domains, according to aspects of the disclosure. The plurality of domains according to some aspects may be stored in an SQL file format. At step 590, the data fabric system 135 may display, via a graphical user interface, one or more graphical depictions of data associated with the plurality of domains as described above with respect to step 390 of FIG. 3.

It should be understood that embodiments in this disclosure are exemplary only, and that other embodiments may include various combinations of features from other embodiments, as well as additional or fewer features. For example, while some of the embodiments above pertain to user data (e.g. patient user data), any suitable activity may be used.

In general, any process or operation discussed in this disclosure that is understood to be computer-implementable, such as the processes illustrated in FIGS. 3 and 5, may be performed by one or more processors of a computer system, such any of the systems or devices in the environment 100 of FIG. 1, as described above. A process or process step performed by one or more processors may also be referred to as an operation. The one or more processors may be configured to perform such processes by having access to instructions (e.g., software or computer-readable code) that, when executed by the one or more processors, cause the one or more processors to perform the processes. The instructions may be stored in a memory of the computer system. A processor may be a central processing unit (CPU), a graphics processing unit (GPU), or any suitable types of processing unit.

A computer system, such as a system or device implementing a process or operation in the examples above, may include one or more computing devices, such as one or more of the systems or devices in FIG. 1. One or more processors of a computer system may be included in a single computing device or distributed among a plurality of computing devices. A memory of the computer system may include the respective memory of each computing device of the plurality of computing devices.

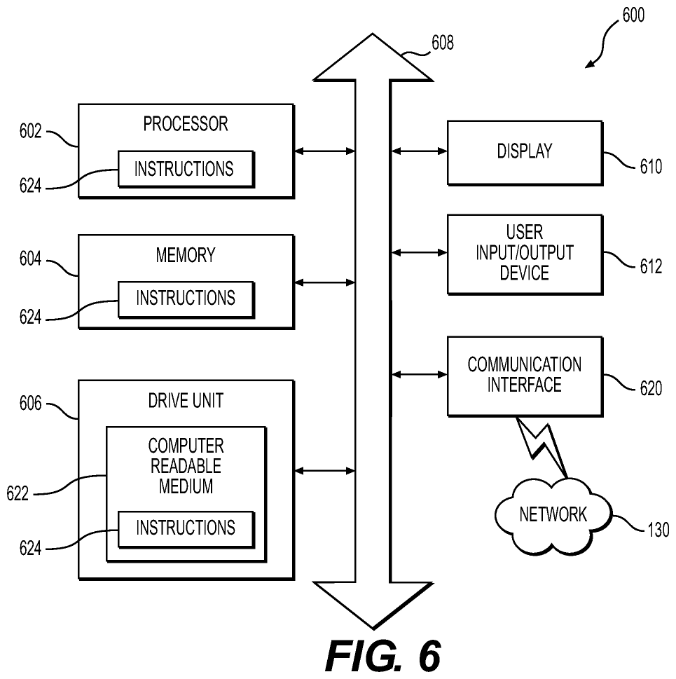

FIG. 6 is a simplified functional block diagram of a computer 600 that may be configured as a device for executing the methods of FIGS. 3 and 4, according to exemplary embodiments of the present disclosure. For example, the computer 600 may be configured as the data fabric system 135 and/or another system according to exemplary embodiments of this disclosure. In various embodiments, any of the systems herein may be a computer 600 including, for example, a data communication interface 620 for packet data communication. The computer 600 also may include a central processing unit (“CPU”) 602, in the form of one or more processors, for executing program instructions. The computer 600 may include an internal communication bus 608, and a storage unit 606 (such as ROM, HDD, SDD, etc.) that may store data on a computer readable medium 622, although the computer 600 may receive programming and data via network communications. The computer 600 may also have a memory 604 (such as RAM) storing instructions 624 for executing techniques presented herein, although the instructions 624 may be stored temporarily or permanently within other modules of computer 600 (e.g., processor 602 and/or computer readable medium 622). The computer 600 also may include input and output ports 612 and/or a display 610 to connect with input and output devices such as keyboards, mice, touchscreens, monitors, displays, etc. The various system functions may be implemented in a distributed fashion on a number of similar platforms, to distribute the processing load. Alternatively, the systems may be implemented by appropriate programming of one computer hardware platform.

Program aspects of the technology may be thought of as “products” or “articles of manufacture” typically in the form of executable code and/or associated data that is carried on or embodied in a type of machine-readable medium. “Storage” type media include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer of the mobile communication network into the computer platform of a server and/or from a server to the mobile device. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

While the disclosed methods, devices, and systems are described with exemplary reference to transmitting data, it should be appreciated that the disclosed embodiments may be applicable to any environment, such as a desktop or laptop computer, an automobile entertainment system, a home entertainment system, etc. Also, the disclosed embodiments may be applicable to any type of Internet protocol.

It should be appreciated that in the above description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those skilled in the art. For example, in the following claims, any of the claimed embodiments can be used in any combination.

Thus, while certain embodiments have been described, those skilled in the art will recognize that other and further modifications may be made thereto without departing from the spirit of the invention, and it is intended to claim all such changes and modifications as falling within the scope of the invention. For example, functionality may be added or deleted from the block diagrams and operations may be interchanged among functional blocks. Steps may be added or deleted to methods described within the scope of the present invention.

The above disclosed subject matter is to be considered illustrative, and not restrictive, and the appended claims are intended to cover all such modifications, enhancements, and other implementations, which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description. While various implementations of the disclosure have been described, it will be apparent to those of ordinary skill in the art that many more implementations are possible within the scope of the disclosure. Accordingly, the disclosure is not to be restricted except in light of the attached claims and their equivalents.

What is claimed is:

1. A computer-implemented method for implementing a data fabric structure, the method comprising:
receiving, by one or more processors, user data associated with a user from an external server via a secure network connection;
storing, by the one or more processors, the user data on a cloud-based data lake;
transmitting, by the one or more processors, the user data to a staging table;
adding, by the one or more processors, to the staging table, user identification data and metadata;
modifying, by the one or more processors, the user data based on a determined correlation between the user data, the user identification data, and metadata to generate modified user data;
extracting, by the one or more processors, relevant data from the modified user data;
formatting, by the one or more processors, the relevant data into atomic data;
generating, by the one or more processors, a plurality of domains based on the atomic data; and
presenting, by the one or more processors, via a graphical user interface, one or more graphical depictions of data associated with the plurality of domains.

2. The computer-implemented method of claim 1, further comprising:
comparing the modified user data and prior user data to determine a difference between the modified user data and the prior user data; and
upon determining that the difference exceeds a predetermined threshold, automatically:
removing the modified user data from the staging table;
refraining from extracting relevant data from the modified user data;
transmitting an error notification to an entity associated with the external server; and
extracting relevant data from the prior user data.

3. The computer-implemented method of claim 2, wherein:
the modified user data includes a quantity indicating a number of members associated with a provider; and
comparing the modified user data and the prior user data includes comparing a number of members associated with the provider and a prior number of members associated with the provider.

4. The computer-implemented method of claim 2, wherein:
the modified user data includes an amount of paid claims associated with a provider; and
comparing the modified user data and the prior user data includes comparing the amount of paid claims associated with the provider and a prior amount of paid claims associated with the provider.

5. The computer-implemented method of claim 1, wherein the user data comprises one or more of: user institution records, user identification information, or user financial data.

6. The computer-implemented method of claim 1, wherein the user data is in a format of one or more of: an .xls file; a csv file; or a text file.

7. The computer-implemented method of claim 1, wherein the external server is associated with a health insurance company or a hospital.

8. The computer-implemented method of claim 1, wherein the secure network connection includes a secure hypertext transfer protocol (S-HTTP).

9. The computer-implemented method of claim 1, wherein the metadata includes a time stamp associated with a time the user data was stored on the cloud-based data lake.

10. The computer-implemented method of claim 1, wherein the plurality of domains include one or more of: a provider domain; a cms domain; a risk domain; a finance domain; a quality domain; a master data domain; a health plan domain; a member domain; a medical claim domain; a T 1 claim domain; or a lab result domain.

11. The computer-implemented method of claim 1, wherein one of the plurality of domains further includes sub-domains.

12. The computer-implemented method of claim 1, wherein each domain of the plurality of domains is stored in an SQL file format.

13. The computer-implemented method of claim 1, wherein extracting relevant data from the modified user data further comprises extracting relevant data from the modified user data using a trained machine learning model.

14. The computer implemented method of claim 13, wherein the trained machine learning model is trained to extract relevant data from the modified user data based on (i) training relevancy data that includes information regarding prior relevant data extracted from prior modified user data associated with other users and (ii) training user data that includes prior relevant data extracted from prior modified user data, to learn relationships between the training relevancy data and the training user data, such that the trained machine learning model is configured to use the learned relationships to extract modified user data in response to input of the modified user data.

15. A system for implementing a data fabric structure, the system comprising:
at least one memory storing instructions; and
at least one processor executing the instructions to perform a process including:
receiving user data associated with a user from an external server via a secure network connection;
storing the user data on a cloud-based data lake;
transmitting the user data to a staging table;
adding, to the staging table, user identification data and metadata;
modifying the user data based on a determined correlation between the user data, the user identification data, and metadata to generate modified user data;
extracting relevant data from the modified user data;
formatting the relevant data into atomic data;
generating a plurality of domains based on the atomic data; and
presenting, via a graphical user interface, one or more graphical depictions of data associated with the plurality of domains.

16. The system of claim 15, the process further including:
comparing the modified user data and prior user data to determine a difference between the modified user data and the prior user data; and
upon determining that the difference exceeds a predetermined threshold automatically:
removing the modified user data from the staging table;
refraining from extracting relevant data from the modified user data;
transmitting an error notification to an entity associated with the external server; and
extracting relevant data from the prior user data.

17. The system of claim 15, wherein the external server is associated with a health insurance company or a hospital.

18. The system of claim 15, wherein:
the metadata includes a time stamp associated with a time the user data was stored on the cloud-based data lake;
one of the plurality of domains further includes sub-domains;
the secure network connection includes a secure hypertext transfer protocol (S-HTTP); and/or
each domain of the plurality of domains is stored in an SQL file format.

19. The system of claim 15, wherein extracting relevant data from the modified user data further comprises extracting relevant data from the modified user data using a trained machine learning model, wherein the trained machine learning model is trained based on (i) training relevancy data that includes information regarding prior relevant data extracted from prior modified user data associated with other users and (ii) training user data that includes prior relevant data extracted from prior modified user data, to learn relationships between the training relevancy data and the training user data, such that the trained machine learning model is configured to use the learned relationships to extract modified user data in response to input of the modified user data.

20. A computer-implemented method for implementing a data fabric structure, the method comprising:
receiving, by one or more processors, user data associated with a user from an external server via a secure network connection;
storing, by the one or more processors, the user data on a cloud-based data lake;
transmitting, by the one or more processors, the user data to a staging table;
adding, by the one or more processors, to the staging table, user identification data and metadata;
modifying, by the one or more processors, the user data based on a determined correlation between the user data, the user identification data, and metadata to generate modified user data;
comparing, by the one or more processors, the modified user data and prior user data to determine a difference between the modified user data and the prior user data;
upon determining that the difference does not exceed a predetermined threshold, extracting, by the one or more processors, using a trained machine learning model, relevant data from the modified user data, wherein the trained machine learning model is trained to extract relevant data from the modified user data based on (i) training relevancy data that includes information regarding prior relevant data extracted from prior modified user data associated with other users and (ii) training user data that includes prior relevant data extracted from prior modified user data, to learn relationships between the training relevancy data and the training user data, such that the trained machine learning model is configured to use the learned relationships to extract modified user data in response to input of the modified user data;
upon determining that the difference does exceed a predetermined threshold, automatically: removing the modified user data from the staging table; transmitting an error notification to an entity associated with the external server; and extracting relevant data from the prior user data;
formatting, by the one or more processors, the relevant data into atomic data;
generating, by the one or more processors, a plurality of domains and sub-domains based on the atomic data; and
displaying, by the one or more processors, via a graphical user interface, one or more graphical depictions of data associated with the plurality of domains and sub-domains.
